(19) United States
(12) Patent Application Publication
(10) Pub. No.: US 2005/0137474 A1
(43) Pub. Date: Jun. 23, 2005

(54) Method for measuring intraocular pressure

(76) Inventor: Antti Kontiola, Helsinki (FI)

Correspondence Address: SHOEMAKER AND MATTARE, LTD, 10 POST OFFICE ROAD - SUITE 110, SILVER SPRING, MD, 20910, US

(21) Appl. No.: 11/007,596

(22) Filed: Dec. 9, 2004

Related U.S. Application Data
(63) Continuation of application No. PCT/FI03/00490, filed on Jun. 17, 2003.

(30) Foreign Application Priority Data
Jun. 17, 2002 (FI) FI20021172

(51) Int. Cl. A61B 3/16

(52) U.S. Cl. 600/398; 600/399; 600/405

(57) ABSTRACT

A method for measuring intraocular pressure includes a probe supported and located in a suitable case, means for giving the probe a specific velocity, and means for processing and displaying the measurement data and controlling operations. According to the method, the probe is given a specific, relatively large initial acceleration, by feeding a voltage/current pulse to the means, particularly a coil, the velocity of the probe is detected using the second means, particularly a coil, on the basis of the data obtained from which, the magnitude of the voltage/current pulse is reduced, in order to reduce and stabilize the acceleration of the probe, the voltage/current pulse is maintained up until the moment of impact of the probe with the eye, or even after the moment of impact, and the impact of the probe is detected with the aid of the coil, in order to calculate and obtain the measurement result, and if desired, the probe is retracted to its departure position within the apparatus, by using the voltage fed to the coil.

### REFERENCE TO EARLIER APPLICATIONS

This application is a continuation-in-part of PCT/FI03/00490, filed Jun. 17, 2003, which claims priority of Finnish application 20021172, filed Jun. 17, 2002.

### BACKGROUND OF THE INVENTION

The present invention relates to a method for measuring intraocular pressure. The method and the apparatus used for the same are based on an understanding of the laws governing the impact of an object with the eye.

Intraocular pressure is generally measured using a tonometer, which is placed on the surface of the cornea and which measures its elasticity using various methods (Goldmann's tonometer, Schiötz's tonometer, etc.). Two of the most commonly used principles are the measurement of the force required to applanate a certain area of the surface of the eye, or the measurement of the diameter of the area that is applanated by a known force. These methods require the patient's co-operation and cannot be applied without general anaesthesia to small children, persons suffering from dementia, or animals.

Methods, such as U.S. patent publications, U.S. Pat. Nos. 5,148,807, 5,279,300, and 5,299,573, in which the surface of the cornea is not touched, the intraocular pressure being measured instead with the aid of a water or air jet, or various kinds of waves, have also been developed. These methods are technically complex and thus expensive. Meters operating on the air-jet principle are widely used by opticians, but their cost has prevented them from being more extensively used by general practitioners.

U.S. Pat. No. 5,176,139 also discloses a method, in which a freely-falling ball is dropped onto the eyelid and the height of the ball's rebound is measured.

It is also known from Finnish patent application 973094 an apparatus which is based on the fact that a probe is forwarded with a certain speed to contact the surface of the eye, wherefrom it will rebounded. The movements of the probe can be the basis for calculation of the intraocular pressure.

The present invention is intended to achieve an improved method specifically when using the latter quite good apparatus. The intention is also, however, to retain the apparatus's simple, economical, and precisely measuring basic construction, by means of which intraocular pressure can also be measured in patients incapable of co-operating, who can be restrained only momentarily. In addition, the meter is also suitable for extensive screening campaigns, as measurement is rapid and requires neither a local anaesthetic nor specially trained operators. It is also intended to permit home monitoring for patients with intraocular pressure complaints.

The above and other benefits and advantages of the present invention are achieved by using an apparatus according to the invention, the characteristic features of which are stated in the accompanying Claims.

### BRIEF DESCRIPTION OF THE DRAWINGS

In the following, the invention is examined with reference to the accompanying drawings, showing the apparatus used in connection of the method according to the invention, in which drawings certain well regarded properties of the invention are presented. Thus:

FIG. 1 shows one version of a practical apparatus, in order to provide a general view of it;

FIG. 2 shows a vertical cross section of the above, rotated to an angle of 90 degrees to the above;

FIG. 3 shows a more detailed picture of the essential component of the invention, by means of which the probe is launched towards the eye.

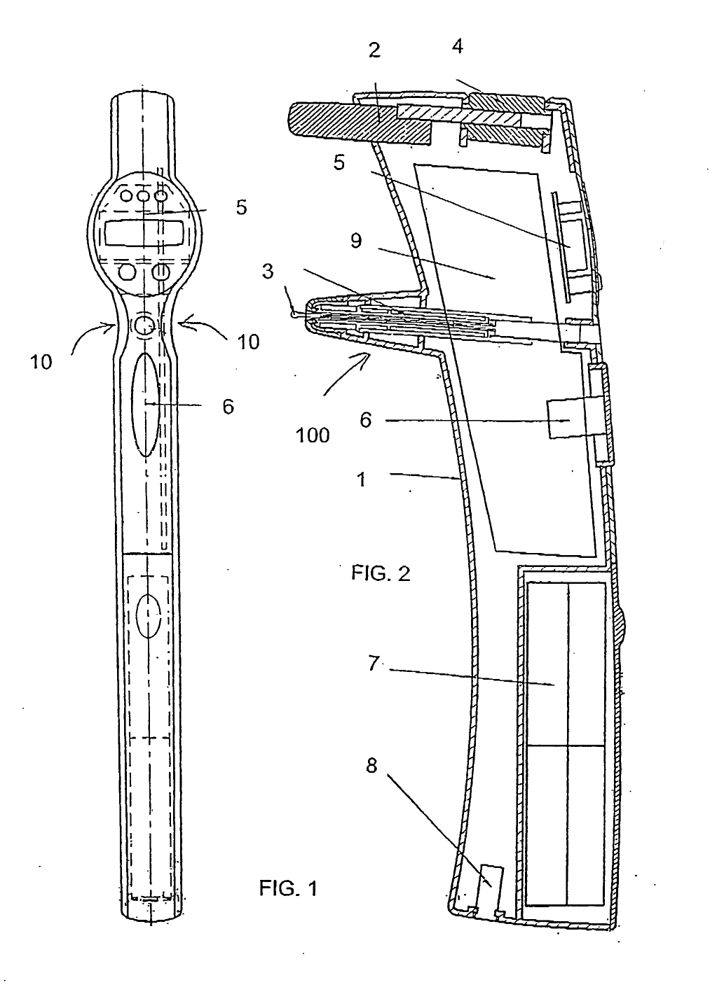

Thus, FIG. 1 shows, as stated above, one practical embodiment of an apparatus applying the principle according to the invention, while FIG. 2 shows a cross section of it. These two figures can be used together to illustrate the general principle and construction.

### DETAILED DESCRIPTION OF THE PREFERRED EMBODIMENTS

The apparatus is formed of a case component 1 made of a suitable material, inside of which all the components that are essential for measurement are fitted. The case or body component 1 is essentially elongated and includes at its upper end a forehead support 2, which is intended to adjust the distance from which the probe 3 impacts the eye being measured. The forehead support 2 is specifically adjustable, one comfortable way of arranging adjustability being to use an adjustment wheel 4, which can be easily rotated with a finger.

The apparatus includes a display and control component 5, which is particularly a liquid-crystal panel, in which the measurement result is displayed, and the related control buttons, etc, which are required at various times. Reference number 6 marks the operating switch, which, when pressed, releases the probe 3 towards the eye.

Operating power for the apparatus comes from dry cells or batteries 7, while the apparatus can additionally have a socket 8, to which an external recharging device or power supply can be connected.

The electronics of the apparatus according to the invention are assembled on a circuit board 9, which is shown schematically.

FIG. 1 clearly shows one property of the apparatus according to the invention that makes it more comfortable and accurate to use. This is the narrowed part, incorporated in the apparatus next to the probe 3. These narrowings are marked with the reference number 10. They are intended to allow patients performing self measurement to be able to see the coloured part of the eye through a mirror, on either side of the narrowing, so that it is easy for them to use this to align the measuring apparatus accurately, thus ensuring that the probe will strike the desired point on the surface of the eyeball. As can be seen, the narrowings 10 are located next to the probe 3.

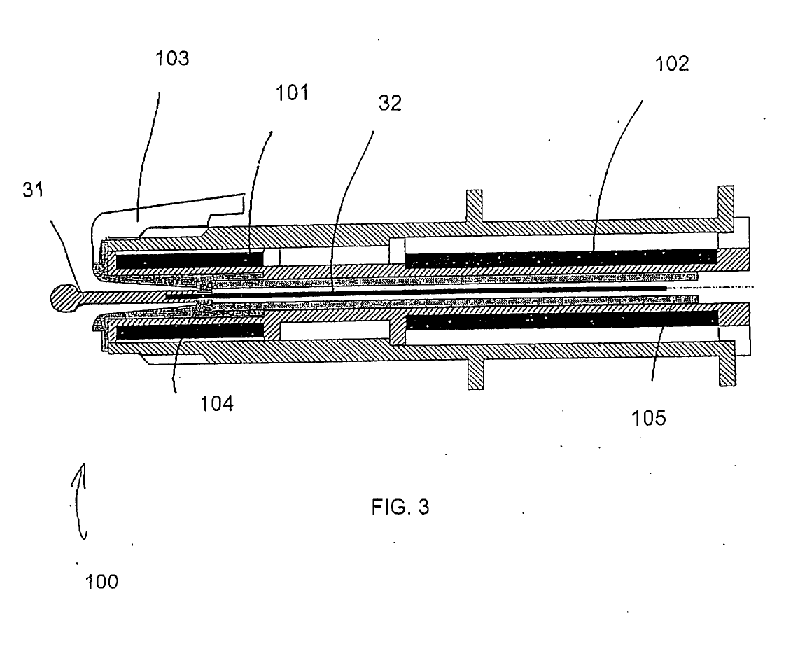

FIG. 3 shows the totality, marked with the reference number 100, relating to the launch of the probe of the apparatus according to the invention, the recording of the measurement, and other similar operations. Thus, according to one well regarded embodiment, the totality 100 includes two coils 101 and 102, the frontmost of which is intended to provide the probe 3 with the necessary velocity. The rearmost coil 102, on the other hand, is related to various measurement and settings functions.

An inner tube 105 is held in place by an inner sleeve 104, which lies around the probe and is secured by means of a separate plug 103 particularly equipped with a screw attachment. The inner sleeve and the inner tube can be changed. Naturally, the probe 3 can be changed while over time the inner sleeve and inner tube may also collect enough extraneous material for this to interfere with measurement in an apparatus with such small tolerances. As cleaning the sleeve and tube is difficult, it will then be easier to change them. If desired, the inner sleeve and the inner tube can be manufactured as a single integrated component.

According to one well regarded embodiment, the probe can be formed of a non-magnetic point part 31 and a shaft part 32 of steel or other similar material.

The point part 31 is preferably manufactured from a plastic material, for reasons of both manufacturing technique and manufacturing cost. Another characteristic of the probe is that a shoulder is formed where the point joins the shaft and is made to rest on the edges of the opening of the inner sleeve 104, through which the shaft part 32 runs. This positions the probe very precisely for the start of the measurement event. For reasons of hygiene, the point part of the probe is generally clearly outside the apparatus in the starting position.

The non-magnetic point part of the probe is in such a ratio to the magnetic shaft part that the shaft extends for a certain distance inside the coil 101, preferably, for example, almost halfway into the coil, counting from its front, as shown in FIG. 3. The voltage fed to the coil induces a pushing force in the probe, causing it to move towards the eye.

Earlier, the probe had the problem that, under certain conditions, the eyelashes tended to catch on the front of the probe. The point part of the probe has now been given a very round shape, thus preventing catching.

The simplified operation of the apparatus is as follows. Power is supplied to the front coil 101, causing the probe to begin moving and to impact the eye. The probe 3 rebounds and the movements, which take place in a manner depending on the intraocular pressure, are recorded with the aid of the rear coil 102, taken to a suitable data-processing unit to be processed particularly by a microprocessor, and the result is displayed by the display device 5. The coil 101 can operate in pulses, or power can be connected to the coil for the entire duration of the measurement, so that the power is cut off only when it is intended to pull the probe back inside.

The stability of the measurement is adjusted in many ways, for example, by triggering the movement of the probe always using a constant voltage, which does not alter when the voltage of the dry cells drops. The constant voltage can be adjustable, making it possible to adjust the launch speed of the probe. The starting position of the probe 3 is also made constant in a suitable manner, for example, by having the shaft of the probe rest on its starting position in the inner sleeve 103. One way of ensuring the starting position is described above. Other ways can also be used.

Many different ways can be used to ensure that the probe remains stationary. One example is that when the power to the apparatus is switched on, the rear, measuring coil 102 holds the probe stationary by magnetic force. A function that throws the probe out after measurement, can also be added to the apparatus. This can particularly be performed manually, by pressing a button. The retaining forces need not be continuous, instead, the apparatus can include a detection function, which, for instance, which monitors the probe's position and pulls a probe, which is trying to detach itself, back into place only when necessary.

As described above, the voltage fed to the coil 101 to perform the actual measurement causes the probe to start moving. The rear coil 102 detects the speed of the probe continuously, the general principle being that the probe accelerates rapidly at the start, then, as the journey continues, a change in the voltage/current supplied to the coil reduces the acceleration to a constant, relatively low level, intended to maintain even motion. The input voltage/current of the coil 101 is preferably on for essentially the whole time that the probe proceeds towards the eye and impacts it, but can also be cut off at, or near to, the moment of impact.

According to yet another preferable embodiment, immediately after the impact has occurred and the measurement result has been obtained, the rear coil 102 is activated and pulls the probe back to the starting position. This ensures that the probe does not detach due to the movements of the meter or for other reasons, but can be cleanly removed from its place and thrown into the garbage.

The retraction of the probe can also be activated on the basis of time. This is because, if the probe has not struck the measurement object within a predetermined time, it can be assumed that the measurement has failed, in which case the retraction is activated.

As stated, the core of the apparatus according to the invention is a microprocessor. It is easy to use this to also perform other control operations than those referred to above. In a known manner, the microprocessor can have the task of monitoring the measurement results and making error messages according to how a measurement result differs from the values to which the system should tend, according to data recorded for the use of the microprocessor. Monitoring and calculation functions of this kind are usual in microprocessor-based devices.

As the probe is light and the velocities used are low, there is no danger of damage to the eye. The method can also be applied when the eyelid is closed. The low velocity and small mass of the probe eliminate the need to anaesthetize the eye under any measurement conditions. If necessary, the meter can be calibrated with different standards, by comparing the results with those obtained by other methods, or by using experimental methods.

As has clearly been demonstrated by the above, the apparatus according to the invention has several properties, by means of which ease of use, accuracy, and reliability can be improved and maintained. Many variations are possible while remaining within the scope of the inventive idea and the accompanying Claims. For example, instead of the front coil 101 giving the probe 3 launching power, it is also possible alternatively to use the rear coil 102, without this altering the characteristics of the apparatus. The physical properties of the coils are always selected as required.

An alternative variation that can also be presented is one, in which the magnetic-material part of the probe, marked with the reference number 32, remains permanently inside the apparatus and is prevented from coming out, by making its rear end wider, thus preventing it from coming out, or in some other suitable manner. The front part, which attaches to the rear part 32, would then be replaceable, due to the demands of hygiene. If desired, the positioning of the components could be set up to take place from the other side of the apparatus to the probe side. In other ways the construction would correspond to that described above.

Particularly a microprocessor-based apparatus can perform many functions, which have previously been impossible. Thus it is possible to perform corrections to the measurement results obtained, in which different variables are measured and observed. Thus, the measured variables could be the impact time of the probe, the time the probe is stationary, and the rebound time, while it is also possible to observe velocities and decelerations, and make various averages and ratios, etc. of them. One correction method is such in which the intraocular pressure is corrected upwards, in proportion to the inelasticity of the impact.

1. A method for measuring intraocular pressure with an apparatus comprising a probe supported and located in a suitable case, means for giving the probe a specific velocity, and means for processing and displaying measurement data and controlling operations, comprising:
feeding a voltage/current pulse to the means for giving the probe a specific velocity, thereby providing the probe with a specific, relatively large initial acceleration;
observing a velocity of the probe using the means for giving the probe a specific velocity on the basis of data obtained from which, or alternatively after a constant time;
reducing a magnitude of the voltage/current pulse in order to reduce and stabilize the acceleration of the probe;
maintaining the voltage/current pulse until a moment of impact of the probe with an eye, or even after the moment of impact; and
observing the impact of the probe with the aid of the means for giving the probe a specific velocity in order to calculate and obtain measurement data.

2. A method according to claim 1, wherein the probe has a rear part made of a magnetic material and a front part, attached thereto in a manner forming a shoulder, made of non-magnetic material and with a round-shaped point, so that a joint between the front part and the rear part is located, at the start of a measurement, inside the means for giving the probe a specific velocity at a distance from a front thereof.

3. A method according to claim 1, wherein the probe is held in place, except for during a measurement event, with the aid of the means for giving the probe a specific velocity, when power is switched on in the apparatus.

4. A method according to claim 1, wherein a narrowing is made in the case of the apparatus, essentially next to the probe, which is intended to permit parts of an eye being measured to be seen, in order to ensure accuracy.

5. A method according to claim 1, wherein a replaceable inner tube is located in the apparatus, inside of which the rear part of the probe is intended to be placed, and an inner sleeve for holding the inner tube in place.

6. A method according to claim 2, wherein the shoulder between the front part and the rear part of the probe is placed, at the start of a measurement, against an opening in the inner sleeve, which receives an edge of the rear part of the probe.

7. A method according to claim 1, further comprising correcting the measurement data based on an algorithm.

8. A method according to claim 1, further comprising correcting the measurement data according to how much kinetic energy is lost in an impact and rebound of the probe.

9. A method according to claim 1, wherein the means for giving the probe a specific velocity comprises a coil.

10. A method according to claim 1, further comprising retracting the probe back to a departure position within the apparatus by using the voltage fed to the means for giving the probe a specific velocity.
